(19) United States
(12) Patent Application Publication
(10) Pub. No.: US 2012/0164708 A1
(43) Pub. Date: Jun. 28, 2012

(54) STABILIZED ENZYME COMPOSITIONS

(76) Inventors: Willem Bijleveld, Den Hoorn (NL); Rudolf Van Der Pol, Schiedam (NL); Roeland Christiaan Vollinga, Woerden (NL)

(21) Appl. No.: 13/382,344

(22) PCT Filed: Jun. 15, 2010
(86) PCT No.: PCT/EP10/58410

§ 371 Date: Mar. 16, 2012

(30) Foreign Application Priority Data
Jul. 9, 2009 (EP) 09165058.0

(51) Int. Cl. C12N 9/96

(52) U.S. Cl. 435/188

(57) ABSTRACT

The present invention relates to a composition comprising an enzyme and octanol. Additionally, the present invention relates to a composition comprising a transition metal ion.

### FIELD OF THE INVENTION

The present invention relates to a composition comprising an enzyme and octanol. Additionally, the present invention relates to a composition comprising a transition metal ion.

### BACKGROUND OF THE INVENTION

The complex chemical structure of enzymes, displaying many different functional groups, not only gives enzymes their unprecedented specificity and reactivity in catalyzing a wide range of conversions, but also is the reason that enzymes are relatively labile compounds. Clearly this phenomenon is the origin of the fact that studies for optimizing enzyme stability are continuously ongoing resulting in a multitude of often specific solutions to a general problem.

Enzymes may be destabilized by unfolding of the three-dimensional structure of the enzyme or by chemical degradation. De-stabilization can easily occur from contact with polar solvents, microbial attack, electrolytes, surfactants, temperature and extreme pH. In order to compensate loss of enzyme activity during periods of storage, formulators may use excess enzymes in liquid enzymatic compositions. However, this an unfavorable solution as enzymes are relatively expensive formulation ingredients. This problem may be overcome by adding stabilizers. Materials that have been used for stabilizing enzymes include various organic and inorganic compounds such as polyols, carboxylic acids, carboxylic acid salts, carboxylic acid esters, and sugars; calcium salts; boron compounds, and various combinations thereof. Protein extracts can also be used to stabilize enzymes through inhibition of the enzyme.

Nevertheless, due to the wide variety of enzymes alternative solutions to the problem of enzyme de-stabilization are still required and will be required in the future.

### DETAILED DESCRIPTION OF THE INVENTION

In a first aspect if the invention there is disclosed a composition comprising an enzyme and octanol. Such a mixture surprisingly displays enhanced stability as compared to the same mixture without octanol. Preferably said octanol is 1-octanol albeit that also isomers such as 2-octanol, 3-octanol, 2-methyl-1-heptanol, 3-methyl-1-heptanol display similar characteristics. The preferred amount of octanol in the composition is from 0.05% to 15% by weight of the total composition, more preferably from 0.1% to 5% by weight of the total composition.

In one embodiment of the first aspect of the present invention, the enzyme is a hydantoin racemase. Polypeptides with hydantoin racemase activity, also called hydantoin racemases, are known in the art. They have been found in a variety of organisms, for instance WO 01/23582 describes a hydantoin racemase from Arthrobacter aurescens (DSM 3747) and JP 04271784 describes a hydantoin racemase from Pseudomonas NS 672 (Watabe et al., J. Bact. 174, 3461-3466 (1992)). Hydantoin racemase have also been described in Sinorhizobium meliloti (acc. nr. CAC 47181, Capela et al., Proc. Natl. Acad. Sci. 98, 9877-9882 (2001)), in Microbacterium liquefaciens (acc. nr. CAD 32593, EP 1188826), and in Agrobacterium tumefaciens strain C58 (acc. nrs. AAL 45498, AAK 88746 and AAK 90298, Las Heras-Vazquez et al., Biochem. Biophys. Res. Commun. 303, 541-547 (2003), Wood et al., Science 294, 2317-2323 (2001) and Hinkle et al., NCBI database, Complete Genome Sequence of Agrobacterium tumefaciens C58 (Rhizobium radiobacter C58), the Causative Agent of Crown Gall Disease in Plants. Direct Submission, submitted Aug. 14, 2001). Isolated polypeptides that exhibit hydantoin racemase and that are void of substrate inhibition have been described in WO 2003/100050. Not unusually hydantoin racemase implies the presence of more than one enzyme such as a hydantoinase and a racemase. It has been found that the present invention also applies to mixtures comprising additional enzymes such carbamoylases.

In a second embodiment the present invention provides a composition comprising an enzyme, an octanol and a transition metal ion. The combination of an enzyme and a metal per se is known. As a matter of fact, a certain class of enzymes, i.e. the metalloenzymes, can only function by virtue of the presence of a metal. Metalloenzyme is a generic term for an enzyme that contains a metal ion cofactor. Indeed, about one quarter to one third of all enzymes require metals to carry out their functions. The metal ion is usually coordinated by nitrogen, oxygen or sulfur atoms belonging to amino acids in the polypeptide chain and/or a macrocyclic ligand incorporated into the enzyme. The presence of the metal ion allows metalloenzymes to perform functions such as redox reactions that cannot easily be performed by the limited set of functional groups found in amino acids (A. Messerschmidt et al., (2001) Handbook of Metalloproteins; Wiley, ISBN 0-471-62743-7). Usually the amount of these metals, such as transition metals, is quite low such that the concentration in formulations does not exceed 1-100 μmol/kg. As a matter of fact, higher concentrations are quite often toxic to the enzyme. It has surprisingly been found that certain, relatively high, concentrations of transition metal have a stabilizing effect on enzymes. Thus, in a composition comprising an enzyme, a concentration of transition metal ion ranging from 2 mmol/kg to 100 mmol/kg leads to enhanced enzyme stability. Preferably said transition metal is present in a concentration ranging from 2.5 mmol/kg to 50 mmol/kg, more preferably from 3 mmol/kg to 25 mmol/kg. Preferably the transition metal of the present invention is cobalt or manganese.

In the context of the present invention, the term transition metal (sometimes also called a transition element) refers to an element whose atom has an incomplete d sub-shell, or which can give rise to cations with an incomplete d sub-shell. This definition corresponds to groups 3 to 11 of the periodic table.

In a second aspect of the present invention there is provided a method for the preparation of a composition comprising an enzyme and an octanol comprising the addition of octanol following the production of said enzyme. Said production may be a fermentation process, optionally followed by one or more downstream processing steps such as concentration, for instance by evaporation, diafiltration, lyophilization, microfiltration, ultrafiltration and similar or other techniques known to the skilled person.

### LEGEND TO THE FIGURE

FIG. 1 shows the influence of the presence of octanol and manganese (Mn²⁺) on the residual activity over time of L-hydantoinase from Escherichia coli RV308. The Y-axis represents the residual activity in % relative to the activity at start which is set at 100%. The X-axis represents the time (h) of incubation. Legend: ⋄=blank (no added Mn²⁺ or octanol); Δ=1 mM Mn²⁺; ▴=5 mM Mn²⁺; □=10 mM Mn²⁺; ◯=octanol; =octanol+1 mM Mn²⁺; ▪=octanol+5 mM Mn²⁺; □=octanol+10 mM Mn²⁺. It can be seen that addition of octanol results in an increase of residual activity compared to the blank sample. Combination with 1 mM, 5 mM and 10 mM Mn²⁺, which in itself also has an effect on stabilizing the enzyme activity, further enhances the positive contribution of octanol.

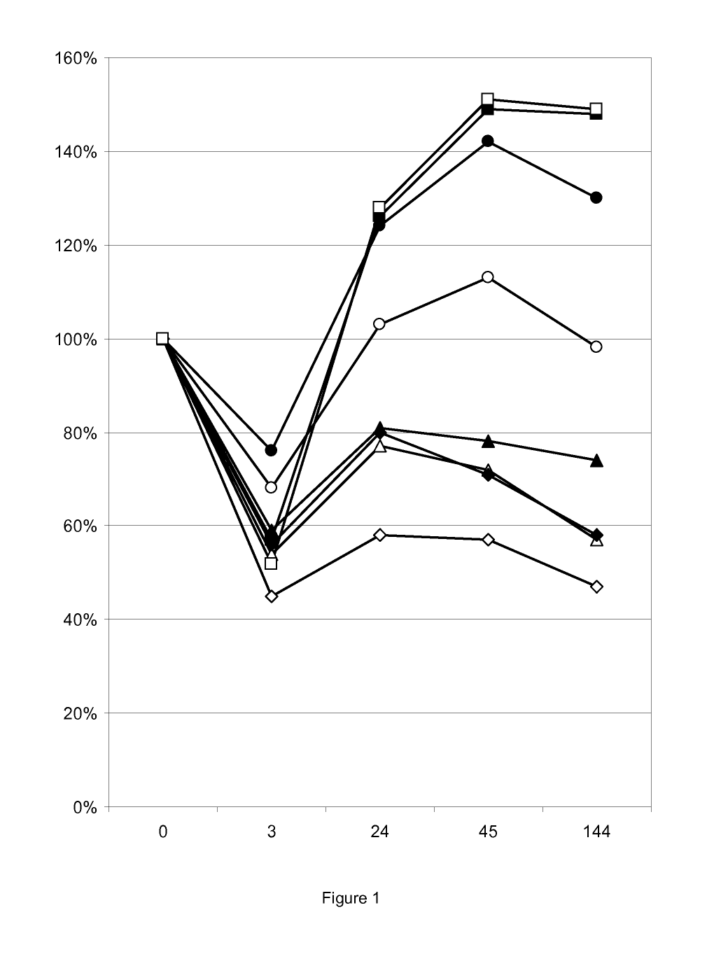

### EXAMPLES

### Materials and Methods

Unless indicated otherwise, all molecular techniques employed were essentially performed according to Maniatis et al. (J. Sambrook, E. F. Fritsch, T. Maniatis. Molecular Cloning 2nd edition. CSH Press).

Protocol for Transformation of pKECaroP-hvu1 Construct into Escherichia coli RV308
Thaw Escherichia coli RV308 aliquots (200 μl, super competent) on ice
Add 15 μl LR reaction mix (see above)
Incubate 30 minutes on ice
Heat shock 1 minute 42° C.
Cool cells 2 minutes on ice
Add 1 ml LB medium (5 g/l NaCl, 5 g/l yeast extract, 10 g/l tryptone)
Incubate 1 hour 37° C.
Plate on LB agar plates supplemented with kanamycine (5 g/l NaCl, 5 g/l yeast extract, 10 g/l tryptone, 15 g/l agar, 50 mg/l kanamycine)
Incubate 24 hours 28° C.
Isolate single colonies
   Protocol for Expression of Hvu Genes in Escherichia coli RV308

Single clones from the transformation (see above) were used to inoculate 5 ml of 2×TY media (10 g/l yeast extract, 16 g/l tryptone, 5 g/l NaCl) supplemented with 0.05 g/l kanamycine and 1 mM MnCl₂ or CoCl₂, respectively. The culture was incubated at 28° C. and 150 rpm for 24 hours and then used for inoculation of 100 ml 2×TY media supplemented with 0.05 g/l kanamycine and 1 mM MnCl₂ or CoCl₂, respectively. The cultures were again incubated for 24-28 hours under conditions previously mentioned and subsequently harvested by centrifugation (20 minutes, 5000 rpm, 4° C.). The cell pellet was resuspended in 5 ml Tris-HCl (100 mM, pH 7), centrifuged again (20 minutes, 5000 rpm, 4° C.) and the cells were frozen at −20° C.

### Analysis Methods

### Hydantoinase Activity Assay

Unit definition: One unit of hydantoinase activity is defined as the amount of enzyme producing 1 μmol of N-carbamoyl phenylalanine per minute at pH 8.0 and 40° C.

Substrate: 100 mM D/L-phenylalanine hydantoine suspension in 130 mM TRIS/HCl buffer pH 8.0 also containing 1.43 mM MnCl₂.

Sample pre-treatment: One gram of sample is suspended in 10 mL 130 mM TRIS/HCl buffer pH 8.0 also containing 1.43 mM MnCl₂. After mixing, the suspension is diluted to approximately 0.9 U/mL with the same buffer. Samples are kept on ice before use. The linear range of this method is from 0.16 to 1.62 U/mL

Assay: 2.1 mL substrate suspension is brought in a reaction tube and subsequently pre-heated for 10 minutes in a 40° C. water bath. The reaction is started by adding 100 μL of sample and mixing. A substrate blank is included by incubating the substrate with 100 μL buffer instead of sample. After 30 minutes the enzymatic reaction is stopped by adding 400 μL 1 M HCl solution followed by mixing and subsequent cooling in ice water. The reaction mixture is filtered over a 0.45 μm filter. The clear solution is transferred into a HPLC injection vial.

Standards: 1 mM N-carbamoyl-L-phenylalanine and L-phenylalanine.

HLPC analysis of reaction mixture and standards:
Column: Xbridge® phenyl 5 μm, 4.6×150 mm, Waters
Detector: UV@220 nm
Flow rate: 1.2 mL/min
Injection volume: 20 μl
Sample tray temp.: 10° C.
Column temp.: ambient
Run time: 20 minutes
Mobile phase A: 40 mM phosphate buffer; pH 5.2/Acetonitrile (98/2 (v/v))
Mobile phase B: 40 mM phosphate buffer; pH 5.2/Acetonitrile (70/30 (v/v))
Gradient

| | Mobile phase | Mobile phase |
|---|---|---|
| Time [min] | A [%] | B [%] |
| 0 | 100 | 0 |
| 13 | 0 | 100 |
| 15 | 100 | 0 |
| 20 | 100 | 0 |

Retention times (may differ depending on the HPLC system used): 3.40 minutes: L-phenylalanine; 5.17 minutes: N-carbamoyl-L-phenylalanine; 9.96 minutes: substrate phenylalanine-hydantoin.

Calculation: The response factors for 1 mM of the standards N-carbamoyl-L-phenylalanine and L-phenylalanine are calculated using the following formulas:

$${RF}_{N \text{-} cpa} = \frac{Peak {area}_{N \text{-} cpa} \times M {W}_{N \text{-} cpa} \times Vk \times {Df}_{N \text{-} cpa} \times 100}{{W}_{N \text{-} cpa} \times {P}_{N \text{-} cpa} \times 1000}$$

$${RF}_{Phe} = \frac{Peak {area}_{Phe} \times M {W}_{Phe} \times Vk \times {Df}_{Phe} \times 100}{{W}_{Phe} \times {P}_{Phe} \times 1000}$$

### Where:

RF_{N-cpa}=Response Factor of 1 mM N-carbamoyl-phenylalanine [mAU·min·L/mmol]
RF_{Phe}=Response Factor of 1 mM phenylalanine [mAU·min·L/mmol]
Peak area_{N-cpe}=Peak area N-carbamoyl-phenylalanine [mAU·min]
Peak area_{Phe}=Peak area phenylalanine [mAU·min]
Vk=Flask volume of standard solution [mL]
Df_{N-cpa}=Total dilution factor of N-carbamoyl-phenylalanine standard solution [mL]
Df_{Phe}=Total dilution factor of phenylalanine standard solution [mL]
W_{N-cpa}=Weight of N-carbamoyl-phenylalanine [mg]
W_{Phe}=Weight of phenylalanine [mg]
P_{N-cpa}=Purity of N-carbamoyl-phenylalanine [%]
P_{Phe}=Purity of phenylalanine [%]
MW_{N-cpa}=Molecular weight N-carbamoyl-phenylalanine (208 g/mol)
MW_{Phe}=Molecular weight phenylalanine (165.19 g/mol)

The hydantoinase activity is calculated using the following formula:

$$U / g = \{ \begin{matrix}( \frac{Area {Sample}_{N \text{-} cpa} - Area {blank}_{N \text{-} cpa}}{{RF}_{N \text{-} cpa}} ) + \\ ( \frac{Area {Sample}_{Phe} - Area {blank}_{Phe}}{{RF}_{Phe}} )\end{matrix} \} \times ( \frac{{Df}_{sam} \times Vk \times {V}_{t}}{{V}_{sam} \times t \times W} )$$

### Where:

Area Sample_{N-cpe}=Peak area of N-carbamoyl-phenylalanine of sample
Area blank_{N-cpa}=Peak area of N-carbamoyl-phenylalanine of blank
Area Sample_{Phe}=Peak area of phenylalanine of sample
Area blank_{Phe}=Peak area of phenylalanine of blank
Vₜ=Total reaction volume (mL)
Dfₛₐₘ=Dilution factor sample
Vₛₐₘ=Volume sample (mL)
Vₖ=Flask volume of sample
t=Time of incubation (min)
W=Weight sample (g)

### Carbamoylase Activity Assay

Unit definition: One unit of carbamoylase activity is defined as the amount of enzyme producing 1 μmol of phenylalanine per minute at pH 8.0 and 40° C.

Substrate: 100 mM N-carbamoyl-L-phenylalanine suspension in 130 mM TRIS/HCl buffer pH 8.0 also containing 1.43 mM MnCl₂.

Sample pre-treatment: One gram of sample is suspended in 10 mL 130 mM TRIS/HCl buffer pH 8.0 also containing 1.43 mM MnCl₂. After mixing, the suspension is diluted to approximately 1.5 U/mL with the same buffer. Samples are kept on ice before use.

The linear range of this activity assay is from 0.32 to 3.15 U/mL.

Assay: See hydantoinase assay.

Standards: 1 mM L-phenylalanine.

HLPC analysis of reaction mixture and standard: See hydantoinase assay

Calculation: The response factor for the 1 mM L-phenylalanine standard is calculated using the following formula:

$${RF}_{Phe} = \frac{Peak {area}_{Phe} \times M {W}_{Phe} \times Vk \times {Df}_{Phe} \times 100}{{W}_{Phe} \times {P}_{Phe} \times 1000}$$

### Where:

RF_{Phe}=Response Factor of 1 mM phenylalanine [mAU×min×L/mmol]
Peak area_{Phe}=Peak area phenylalanine [mAU×min]
Vk=Flask volume of phenylalanine standard solution [mL]
Df_{Phe}=Dilution factor of phenylalanine standard solution
P_{Phe}=Purity of phenylalanine [%]
MW_{Phe}=Molecular weight phenylalanine [165.19 mg/mmol]

The carbamoylase activity is calculated using the following formula:

$$U / g = ( \frac{Area {Sample}_{Phe} - Area {blank}_{Phe }}{{RF}_{Phe}} ) \times ( \frac{{Df}_{sam} \times Vk \times {V}_{t}}{{V}_{sam} \times t \times W} )$$

### Where:

Area Sample_{Phe}=Peak area of phenylalanine of sample [mAU×min]
Area blank_{Phe}=Peak area of phenylalanine of blank [mAU×min]
Vₜ=Total reaction volume [mL]
Dfₛₐₘ=Dilution factor sample
Vₖ=Flask volume of sample
Vₛₐₘ=Volume sample [mL]
t=Time of incubation [min]
W=Weight sample [g]

### Racemase Activity Assay

Unit definition: One unit of racemase activity is defined as the amount of enzyme producing 1 μmol of L-phenylalanine-hydantoin from D-phenylalanine-hydantoin per minute at pH 8.0 and 37° C.

Substrate: 10 mM D-phenylalanine-hydantoin solution in 130 mM TRIS/HCl buffer pH 8.0 also containing 0.1 M EDTA. Solution must be made at 37° C.

Sample pre-treatment: One gram of sample is suspended in 10 mL 130 mM TRIS/HCl buffer pH 8.0 also containing 0.1 M EDTA. After mixing, the suspension is diluted to approximately 0.5 U/mL with the same buffer. Samples are kept on ice before use. Linear range of the assay is from 0.19 to 1.16 U/mL.

Assay: 2.0 mL pre-heated substrate solution is brought in a reaction tube in a 37° C. water bath. After 2 minutes the reaction is started by adding 100 μL of sample and mixing. A substrate blank is included by incubating the substrate with 100 μL buffer instead of sample. After 30 minutes the enzymatic reaction is stopped by adding 400 μL 1 M NaOH solution followed by mixing. The reaction mixture is filtered over a 0.45 μm filter. The clear solution is transferred into a HPLC injection vial.

Standards: 1 mM L-phenylalanine-hydantoin and 1 mM N-carbamoyl-L-phenylalanine

HLPC analysis of reaction mixture and standard:
Column, w. precolumn: Chirobiotic T (250 mm×4.6 mm I.D., 5 μm), Astec
Detector: UV@220 nm
Flow rate: 1.5 mL/min
Injection volume: 20 μl
Sample tray temp.: 10° C.
Column temp.: ambient
Run time: 8 minutes, isocratic
Mobile phase: 15 mM ammonium acetate pH 4.1/20% Methanol

Retention times (may differ depending on the HPLC system used): 5.46 minutes: substrate D-phenylalanine-hydantoin; 7.21 minutes: product L-phenylalanine-hydantoin. When hydantoinase is not completely inhibited by EDTA, then peaks of L- and D-carbamoyl-phenylalanine can be visible at approx. 2.8 and 3.5 minutes, respectively.

### Calculation

The response factor for the 1 mM L-phenylalanine standard is calculated using the following formula:

$${RF}_{LPH} = \frac{Peak {area}_{LPH} \times M {W}_{LPH} \times {Vk}_{LPH} \times 100}{{W}_{LPH} \times {P}_{LPH} \times 1000}$$

### Where:

RF_{LPH}=Response Factor of 1 mM L-phenylalanine-hydantoin
Peak area_{LPH}=Peak area L-phenylalanine-hydantoin [mAU×min]
Vk_{LPH}=Flask vol. of L-phenylalanine-hydantoin standard solution [mL]
W_{LPH}=Weight of L-phenylalanine-hydantoin [mg]
P_{LPH}=Purity of L-phenylalanine-hydantoin [%]
MW_{LPH}=Molecular weight L-phenylalanine-hydantoin [190 g/mol]

The response factor for 1 mM of the standard N-carbamoyl-L-phenylalanine is calculated using the following formula:

$${RF}_{LCP} = \frac{Peak {area}_{LCP} \times M {W}_{LCP} \times {Vk}_{LCP} \times 100}{{W}_{LCP} \times {P}_{LCP} \times 1000}$$

### Where:

RF_{LCP}=Response Factor of 1 mM N-carbamoyl-L-phenylalanine
Peak area_{LCP}=Peak area N-carbamoyl-L-phenylalanine [mAU×min]
Vk_{LCP}=Flask vol. of N-carbamoyl-L-phenylalanine standard [mL]
W_{LCP}=Weight of N-carbamoyl-L-phenylalanine [mg]
P_{LCP}=Purity of N-carbamoyl-L-phenylalanine [%]

The racemase activity is calculated using the following formula:

$$U / g = \{ \begin{matrix}( \frac{Area {Sample}_{LPH} - Area {blank}_{LPH}}{{RF}_{LPH}} ) + \\ ( \frac{Area {Sample}_{LCP} - Area {blank}_{LCP}}{{RF}_{LCP}} )\end{matrix} \} \times ( \frac{{Df}_{sam} \times {V}_{k} \times {V}_{t}}{{V}_{sam} \times t \times W} )$$

### Where:

Area Sample_{LPH}=Peak area of L-phenylalanine-hydantoin of sample [mAU×min]
Area blank_{LPH}=Corr. area of L-phenylalanine-hydantoin of blank [mAU×min]
Area Sample_{LCP}=Area of N-carbamoyl-L-phenylalanine of sample [mAU×min]
Area blank_{LCP}=Peak area of N-carbamoyl-L-phenylalanine of blank [mAU×min]
Vₜ=Total reaction volume [mL]
Dfₛₐₘ=Dilution factor sample
Vₛₐₘ=Volume sample [mL]
t=Time of incubation [min]
Vₖ=Flask volume of sample [mL]
W=Weight sample [g]

The corrected peak area of L-phenylalanine-hydantoin of the blank is necessary to correct for the spontaneous racemisation that occurs during the time the samples are in the HPLC and is calculated as follows. The difference of the blanks at the end of the series and start of the series is divided by number of runs between them. This value represents the increase in LPH during each run. This value is added to the value of the first blank, multiplied by the amount of runs between the sample and the first blank.

### Example 1

### Construction of a Clone for co-Expression of L-Hydantoinase, L-Carbamoylase and Hydantoin Racemase in Escherichia coli RV308

The aim was to obtain active coexpression of the L-hydantoinase from Arthrobacter aurescens (HyuH), the L-carbamoylase from Bacillus stearothermophilus (HyuC) and the hydantoin racemase from Agrobacterium radiobacter (HyuA) in the host Escherichia coli RV308 resulting in a production strain for the production of L-amino acids. The sequences of the 3 enzymes are known from the following literature sources:
L-hydantoinase from Abendrodt et al. Biochemistry 41 (27), 8589-8597 (2002);
L-carbamoylase from Battise et al. Appl. Environ. Microbiol. 63(2), 763-766 (1997);
Hydantoin racemase from EP 1506294 B1.

An operon was synthetically prepared according to WO 2008/067981 wherein the three genes of the hydantoin pathway (hyuH, hyuC, hyuA) are separated from each other by spacers containing a ribosomal binding site rbs (Shine-Delgarno Sequence) and a restriction site for further subcloning. The DNA sequences of the enzyme-encoding regions were optimized for expression in Escherichia coli RV308.

The Hyu1 operon was subsequently cloned into an expression vector. The expression vector pKECaro_hyu1 is derived from plasmid pKECtrp (described in WO 00/66751) by replacing the trp promoter==>PenG acylase expression cassette by the aroH promoter==>hyu1 operon. The DNA was transformed into supercompetent Escherichia coli RV308 cells (as described in Material and Methods) and single clones were isolated from the agar plate. The clones were grown in LB medium supplemented with kanamycin (5 g/l NaCl, 5 g/l yeast extract, 10 g/l tryptone, 50 mg/l kanamycin) and plasmid DNA was isolated using the Qiagen Miniprep Kit (following the standard procedure). The accuracy of the constructs was checked by restriction analysis.

### Example 2

### Fermentation of in Escherichia coli RV308 Expressing L-Hydantoinase, L-Carbamoylase and Hydantoin Racemase Activity

Transformed supercompetent Escherichia coli RV308 cells as described in Example 1 were fermented at pH 7.15±0.15 and 27.0±0.5° C. using the fermentation medium outlined in Table 1 wherein glucose and thiamine were fed during the process. The pH was controlled with NH₃ (25%). At the end of the fermentation (approx. 100 h), 1-octanol (4.0 g/kg) and MnSO₄.H₂O (2.4 g/kg) were added after which the broth was cooled to ≦5±1° C.

TABLE 1
Composition of fermentation medium

| | Concentration |
|---|---|
| Component | (g/kg) |
| Yeast extract | 24.6 |
| Citric acid•H₂O | 10.0 |
| K₂HPO₄ | 8.9 |
| FeSO₄•7H₂O | 0.20 |
| MgSO₄•7H₂O | 3.1 |
| CaCl₂ as 25% solution | 4.6 |
| MnSO₄•H₂O | 0.51 |
| (NH₄)₂SO₄ | 5.0 |
| CoCl₂•6H₂O | 0.006 |
| NaMoO₄•2H₂O | 0.004 |
| H₃BO₃ | 0.004 |
| Basildon (antifoam) | 0.15 |
| Glucose | 10.5 |
| Neomycin sulfate | 0.10 |
| Thiamine•HCl | 0.014 |

### Example 3

### Stability L-Hydantoinase, L-Carbamoylase and Hydantoin Racemase at 4° C. with and without Addition of Octanol and Manganese

A sample from the fermentation broth obtained in Example 2 was used for stability testing for the enzymes L-hydantoinase, L-carbamoylase and hydantoin racemase in the absence and presence of octanol and/or Mn²⁺ at three different incubation times. The results are summarized in the below overview.

| | Incuba- | | | |
|---|---|---|---|---|
| | tion | Hydantoinase | Carbamoylase | Racemase |
| Sample | time (h) | (U/mL) | (U/mL) | (U/mL) |
| Series 1 | 4 | 580 | 18 | 26 |
| Series 1 | 25 | 123 | 12 | 18 |
| Series 1 | 48 | 74 | 6.3 | 16 |
| Series 2: + Mn²⁺ | 4 | 619 | 18 | 20 |
| Series 2: + Mn²⁺ | 25 | 303 | 16 | 26 |
| Series 2: + Mn²⁺ | 48 | 258 | 16 | 26 |
| Series 3: + | 4 | 3840 | 20 | 23 |
| octanol + Mn²⁺ | | | | |
| Series 3: + | 25 | 3776 | 20 | 26 |
| octanol + Mn²⁺ | | | | |
| Series 3: + | 48 | 3645 | 18 | 24 |
| octanol + Mn²⁺ | | | | |

### Example 4

### Stability L-Hydantoinase at 4° C. with and without Addition of Octanol and Manganese; end of Fermentation with 1 mM Mn²⁺

A sample from the fermentation broth obtained in Example 2 was used for stability testing for L-hydantoinase in the absence and presence of octanol and/or 1 mM Mn²⁺ at five different incubation times. The results are summarized in the below overview.

| | Residual activity (%) | | | | |
|---|---|---|---|---|---|
| Addition | 0 h | 3 h | 24 h | 46 h | 144 h |
| None | 100 | 45 | 58 | 57 | 47 |
| 1 mM Mn²⁺ | 100 | 54 | 77 | 72 | 57 |
| 5 mM Mn²⁺ | 100 | 59 | 81 | 78 | 74 |
| 10 mM Mn²⁺ | 100 | 56 | 80 | 71 | 58 |
| Octanol | 100 | 68 | 103 | 113 | 98 |
| Octanol + 1 mM Mn²⁺ | 100 | 76 | 124 | 142 | 130 |
| Octanol + 5 mM Mn²⁺ | 100 | 57 | 126 | 149 | 148 |
| Octanol + 10 mM Mn²⁺ | 100 | 52 | 128 | 151 | 149 |

### Example 5

### Stability L-Hydantoinase at 4° C. with and without Addition of Octanol and Manganese; End of Fermentation with 3 mM Mn²⁺

A sample from the fermentation broth obtained in Example 2 was used for stability testing for L-hydantoinase in the absence and presence of octanol and/or 3 mM Mn²⁺ octanol at five different incubation times. The results are summarized below.

| | Residual activity (%) | | | | |
|---|---|---|---|---|---|
| Addition | 0 h | 3 h | 24 h | 46 h | 144 h |
| None | 100 | 48 | 70 | 74 | 50 |
| 1 mM Mn²⁺ | 100 | 56 | 86 | 82 | 57 |
| 5 mM Mn²⁺ | 100 | 74 | 86 | 89 | 62 |
| 10 mM Mn²⁺ | 100 | 60 | 83 | 85 | 58 |
| Octanol | 100 | 94 | 117 | 126 | 110 |
| Octanol + 1 mM Mn²⁺ | 100 | 90 | 134 | 145 | 138 |
| Octanol + 5 mM Mn²⁺ | 100 | 51 | 102 | 162 | 164 |
| Octanol + 10 mM Mn²⁺ | 100 | 50 | 112 | 158 | 165 |

### Example 6

Multilevel Factorial Design Analysis on the Stability of L-Hydantoinase, L-Carbamoylase and Hydantoin Racemase vs Variations in Time, Temperature and Presence or Absence of Octanol, Manganese and Flocculant

A sample from the fermentation broth obtained in Example 2 was used for multilevel factorial design analysis on the stability of L-hydantoinase, L carbamoylase and hydantoin racemase vs variations in time, temperature and presence or absence of octanol, Mn²⁺ and flocculant. The results are summarized in Table 2.
Column A: Random order Column G: Manganese (mM)
Column B: Standard order Column H: Flocculant (g/L)
Column C: Mixture number Column I: Hydantoinase (U/g)
Column D: Time (days) Column J: Carbamoylase (U/g)
Column E: Temperature (° C.) Column K: Hydantoin racemase (U/g)
Column F: Octanol (g/L)

After running a Pareto chart from the above experiments it can be concluded that:
For hydantoinase addition of octanol and Mn²⁺ has a strong positive initial effect, there is a strong positive effect from the interaction between temperature and Mn²⁺ and there are strong negative effects from interaction of octanol/flocculant and Mn²⁺/flocculant. The stability in the presence of octanol/Mn²⁺ at 4° C. is good.
For carbamoylase there is a strong positive effect by addition of Mn²⁺ and there is a strong negative effect from the flocculant. The stability in the presence of octanol/Mn²⁺ at 4° C. is good.

For hydantoin racemase there is a strong negative effect from the flocculant and the stability in the presence of octanol/Mn²⁺ at 4° C. is good.

TABLE 2
Multilevel factorial design analysis on
the stability of L-hydantoinase

| A | B | C | D | E | F | G | H | I | J | K |
|---|---|---|---|---|---|---|---|---|---|---|
| 33 | 1 | 1 | 0 | 4 | 0 | 0 | 0 | 334 | 12.5 | 19.3 |
| 39 | 2 | 1 | 2 | 4 | 0 | 0 | 0 | 140 | 10.7 | 18.8 |
| 31 | 3 | 1 | 4 | 4 | 0 | 0 | 0 | 174 | 12.0 | 18.8 |
| 32 | 4 | 1 | 0 | 20 | 0 | 0 | 0 | 339 | 13.4 | 19.1 |
| 8 | 5 | 1 | 2 | 20 | 0 | 0 | 0 | 192 | 15.4 | 18.5 |
| 11 | 6 | 1 | 4 | 20 | 0 | 0 | 0 | 229 | 15.9 | 18.2 |
| 6 | 7 | 5 | 0 | 4 | 4 | 0 | 0 | 560 | 14.8 | 18.8 |
| 28 | 8 | 5 | 2 | 4 | 4 | 0 | 0 | 404 | 12.7 | 17.6 |
| 30 | 9 | 5 | 4 | 4 | 4 | 0 | 0 | 386 | 14.0 | 15.4 |
| 35 | 10 | 5 | 0 | 20 | 4 | 0 | 0 | 628 | 14.8 | 18.4 |
| 38 | 11 | 5 | 2 | 20 | 4 | 0 | 0 | 461 | 13.1 | 19.6 |
| 29 | 12 | 5 | 4 | 20 | 4 | 0 | 0 | 561 | 11.5 | 13.1 |
| 40 | 13 | 3 | 0 | 4 | 0 | 10 | 0 | 365 | 16.5 | 18.8 |
| 24 | 14 | 3 | 2 | 4 | 0 | 10 | 0 | 244 | 16.6 | 18.3 |
| 14 | 15 | 3 | 4 | 4 | 0 | 10 | 0 | 342 | 17.7 | 17.9 |
| 44 | 16 | 3 | 0 | 20 | 0 | 10 | 0 | 371 | 16.1 | 18.2 |
| 41 | 17 | 3 | 2 | 20 | 0 | 10 | 0 | 449 | 19.2 | 18.4 |
| 16 | 18 | 3 | 4 | 20 | 0 | 10 | 0 | 517 | 19.7 | 18.0 |
| 27 | 19 | 7 | 0 | 4 | 4 | 10 | 0 | 2530 | 18.6 | 18.7 |
| 37 | 20 | 7 | 2 | 4 | 4 | 10 | 0 | 2220 | 18.2 | 17.3 |
| 42 | 21 | 7 | 4 | 4 | 4 | 10 | 0 | 2220 | 18.2 | 18.0 |
| 9 | 22 | 7 | 0 | 20 | 4 | 10 | 0 | 2450 | 18.7 | 20.1 |
| 13 | 23 | 7 | 2 | 20 | 4 | 10 | 0 | 2350 | 15.1 | 15.2 |
| 36 | 24 | 7 | 4 | 20 | 4 | 10 | 0 | 2400 | 13.2 | 14.6 |
| 7 | 25 | 2 | 0 | 4 | 0 | 0 | 1 | 589 | 13.2 | 15.1 |
| 3 | 26 | 2 | 2 | 4 | 0 | 0 | 1 | 391 | 13.4 | 14.1 |
| 45 | 27 | 2 | 4 | 4 | 0 | 0 | 1 | 478 | 11.8 | 14.5 |
| 23 | 28 | 2 | 0 | 20 | 0 | 0 | 1 | 678 | 10.5 | 14.6 |
| 22 | 29 | 2 | 2 | 20 | 0 | 0 | 1 | 336 | 12.5 | 14.4 |
| 4 | 30 | 2 | 4 | 20 | 0 | 0 | 1 | 461 | 13.6 | 14.5 |
| 1 | 31 | 6 | 0 | 4 | 4 | 0 | 1 | 463 | 13.8 | 10.5 |
| 47 | 32 | 6 | 2 | 4 | 4 | 0 | 1 | 427 | 11.3 | 11.6 |
| 21 | 33 | 6 | 4 | 4 | 4 | 0 | 1 | 528 | 11.4 | 11.9 |
| 46 | 34 | 6 | 0 | 20 | 4 | 0 | 1 | 427 | 12.0 | 12.9 |
| 19 | 35 | 6 | 2 | 20 | 4 | 0 | 1 | 549 | 10.2 | 11.9 |
| 2 | 36 | 6 | 4 | 20 | 4 | 0 | 1 | 569 | 8.4 | 12.1 |
| 18 | 37 | 4 | 0 | 4 | 0 | 10 | 1 | 1240 | 15.4 | 16.1 |
| 5 | 38 | 4 | 2 | 4 | 0 | 10 | 1 | 832 | 15.5 | 15.3 |
| 26 | 39 | 4 | 4 | 4 | 0 | 10 | 1 | 660 | 14.6 | 15.0 |
| 25 | 40 | 4 | 0 | 20 | 0 | 10 | 1 | 1160 | 14.3 | 15.5 |
| 12 | 41 | 4 | 2 | 20 | 0 | 10 | 1 | 692 | 15.6 | 17.4 |
| 43 | 42 | 4 | 4 | 20 | 0 | 10 | 1 | 929 | 15.5 | 14.3 |
| 10 | 43 | 8 | 0 | 4 | 4 | 10 | 1 | 1200 | 17.2 | 11.6 |
| 20 | 44 | 8 | 2 | 4 | 4 | 10 | 1 | 1140 | 13.6 | 14.0 |
| 15 | 45 | 8 | 4 | 4 | 4 | 10 | 1 | 1260 | 12.1 | 15.7 |
| 34 | 46 | 8 | 0 | 20 | 4 | 10 | 1 | 1300 | 16.1 | 11.7 |
| 17 | 47 | 8 | 2 | 20 | 4 | 10 | 1 | 1740 | 10.0 | 12.8 |
| 48 | 48 | 8 | 4 | 20 | 4 | 10 | 1 | 1720 | 8.9 | 8.3 |
| Ref | | | | | | | | 304 | 12.4 | 17.3 |

1. Composition comprising an enzyme and octanol wherein the amount of octanol is from 0.1% to 5% by weight of the total composition.

2. A composition according to claim 1 wherein said octanol is 1-octanol.

3. A composition according to claim 1 wherein said enzyme is a hydantoinase.

4. A composition according to claim 3 further comprising a carbamoylase.

5. A composition according to claim 3 further comprising a racemase.

6. A composition according to claim 1 further comprising a transition metal ion.

7. A composition according to claim 6 wherein said transition metal is cobalt or manganese.

8. Method for the production of a composition comprising an enzyme and octanol wherein the amount of octanol is from 0.1% to 5% by weight of the total composition comprising addition of octanol following the production of said enzyme.

9. Method according to claim 8 wherein said octanol is 1-octanol.
